SEQUENCE LISTING (40 sequences)

SEQ ID NO: 1 - DNA, 28 bp, Artificial Sequence
  Primer Oligonucleotide
tcttttcggttagggttaggtaggttgt

SEQ ID NO: 2 - DNA, 47 bp, Artificial Sequence
  Primer Oligonucleotide
gtaatacgactcactatagggagactacaccaatacaaccacatatc

SEQ ID NO: 3 - RNA, 205 nt, Artificial Sequence
  chemically treated RNA
gggagacuacaccaauacaaccacauaucgaucacguacgcccacacccaaccaaucgacgaacucccgacgaaaauaaaaaacgcccuaauccgcauccaacgaauuacacaacuacuucucucuccgcuucccgacccgcacuccgcaauaaaacacaaaaccccgcccaaccgcacaaccuaccuaacccuaaccgaaaaga

SEQ ID NO: 4 - DNA, 30 bp, Artificial Sequence
  Primer Oligonucleotide
tctttttctttgtattaggttggaagtggt

SEQ ID NO: 5 - DNA, 45 bp, Artificial Sequence
  Primer Oligonucleotide
gtaatacgactcactatagggagcccaaataaatcaacaacaaca

SEQ ID NO: 6 - RNA, 299 nt, Artificial Sequence
  chemically treated RNA
gggagcccaaauaaaucaacaacaacaucacgaaaacauuaaauaaaaacuaauaaccaaaaccaauaacuuuacaaaacgaauuccuuccuaacgcucccucguuuuacauaacaaauacgaaauaaacaccucgcgaaaaacgaaccccgcgaaaauaacaucccauuuacuucuuuaaacuauuaaaacucaaccucacaaaucacgcuaaacaauaccaacuaauuccacuuuuccaaaaaauaaaauuacacgaaaaacuaacgaccacuuccaaccuaauacaaagaaaaaga

SEQ ID NO: 7 - RNA, 298 nt, Artificial Sequence
  chemically treated RNA
gggagcccaaauaaaucaacaacaacaucacaaaaacauuaaauaaaaacuaauaaccaaaacaauaacuuuacaaaacgaauuccuuccuaacgcucccucguuuuacauaacaaauacgaaauaaacaccucgcgaaaaacgaaccccgcgaaaauaacaucccauuuacuucuuuaaacuauuaaaacucaaccucacaaaucacgcuaaacaauaccaacuaauuccacuuuuccagaaaauaaaauuacacgaaaaacugacgaccacuuccaaccuaauacaaagaaaaaga

SEQ ID NO: 8 - DNA, 30 bp, Artificial Sequence
  Primer Oligonucleotide
tctttttcatatacgtgtgggtataaaatc

SEQ ID NO: 9 - DNA, 43 bp, Artificial Sequence
  Primer Oligonucleotide
gtaatacgactcactatagggagcaaaaatcaaacaacaacga

SEQ ID NO: 10 - RNA, 25 nt, Artificial Sequence
  RNA fragment after digestion of APC-198 transcript with RNase T1
acuacaccaauacaaccacauaucg

SEQ ID NO: 11 - RNA, 18 nt, Artificial Sequence
  RNA fragment after digestion of APC-198 transcript with RNase T1
cccacacccaaccaaucg

SEQ ID NO: 12 - RNA, 13 nt, Artificial Sequence
  RNA fragment after digestion of APC-198 transcript with RNase T1
aaaauaaaaaacg

SEQ ID NO: 13 - RNA, 10 nt, Artificial Sequence
  RNA fragment after digestion of APC-198 transcript with RNase T1
cccuaauccg

SEQ ID NO: 14 - RNA, 25 nt, Artificial Sequence
  RNA fragment after digestion of APC-198 transcript with RNase T1
aauuacacaacuacuucucucuccg

SEQ ID NO: 15 - RNA, 20 nt, Artificial Sequence
  RNA fragment after digestion of APC-198 transcript with RNase T1
caauaaaacacaaaaccccg

SEQ ID NO: 16 - RNA, 23 nt, Artificial Sequence
  RNA fragment after digestion of APC-198 transcript with RNase T1
cacaaccuaccuaacccuaaccg

SEQ ID NO: 17 - RNA, 27 nt, Artificial Sequence
  RNA fragment after digestion of CDH13 transcript with RNase T1
cccaaauaaaucaacaacaacaucacg

SEQ ID NO: 18 - RNA, 49 nt, Artificial Sequence
  RNA fragment after digestion of CDH13 transcript with RNase T1
aaaacauuaaauaaaaacuaauaaccaaaaccaauaacuuuacaaaacg

SEQ ID NO: 19 - RNA, 75 nt, Artificial Sequence
  RNA fragment after digestion of CDH13 transcript with RNase T1
cccaaauaaaucaacaacaacaucacaaaaacauuaaauaaaaacuaauaaccaaaacaauaacuuuacaaaacg

SEQ ID NO: 20 - RNA, 15 nt, Artificial Sequence
  RNA fragment after digestion of CDH13 transcript with RNase T1
aauuccuuccuaacg

SEQ ID NO: 21 - RNA, 15 nt, Artificial Sequence
  RNA fragment after digestion of CDH13 transcript with RNase T1
aauuccuuccuaacg

SEQ ID NO: 22 - RNA, 18 nt, Artificial Sequence
  RNA fragment after digestion of CDH13 transcript with RNase T1
uuuuacauaacaaauacg

SEQ ID NO: 23 - RNA, 18 nt, Artificial Sequence
  RNA fragment after digestion of CDH13 transcript with RNase T1
uuuuacauaacaaauacg

SEQ ID NO: 24 - RNA, 14 nt, Artificial Sequence
  RNA fragment after digestion of CDH13 transcript with RNase T1
aaauaaacaccucg

SEQ ID NO: 25 - RNA, 14 nt, Artificial Sequence
  RNA fragment after digestion of CDH13 transcript with RNase T1
aaauaaacaccucg

SEQ ID NO: 26 - RNA, 56 nt, Artificial Sequence
  RNA fragment after digestion of CDH13 transcript with RNase T1
aaaauaacaucccauuuacuucuuuaaacuauuaaaacucaaccucacaaaucacg

SEQ ID NO: 27 - RNA, 56 nt, Artificial Sequence; identical to SEQ ID NO: 26
  RNA fragment after digestion of CDH13 transcript with RNase T1

SEQ ID NO: 28 - RNA, 48 nt, Artificial Sequence
  RNA fragment after digestion of CDH13 transcript with RNase T1
cuaaacaauaccaacuaauuccacuuuuccaaaaaauaaaauuacacg

SEQ ID NO: 29 - RNA, 32 nt, Artificial Sequence
  RNA fragment after digestion of CDH13 transcript with RNase T1
cuaaacaauaccaacuaauuccacuuuuccag

SEQ ID NO: 30 - RNA, 16 nt, Artificial Sequence
  RNA fragment after digestion of CDH13 transcript with RNase T1
aaaauaaaauuacacg

SEQ ID NO: 31 - RNA, 11 nt, Artificial Sequence
  RNA fragment after digestion of CDH13 transcript with RNase T1
aaaaacuaacg

SEQ ID NO: 32 - RNA, 23 nt, Artificial Sequence
  RNA fragment after digestion of CDH13 transcript with RNase T1
accacuuccaaccuaauacaaag

SEQ ID NO: 33 - RNA, 23 nt, Artificial Sequence
  RNA fragment after digestion of CDH13 transcript with RNase T1
accacuuccaaccuaauacaaag

SEQ ID NO: 34 - RNA, 19 nt, Artificial sequence
  chemically treated RNA after digestion with RNase T1
caaaaaucaaacaacaacg

SEQ ID NO: 35 - RNA, 16 nt, Artificial Sequence
  chemically treated RNA after digestion with RNase T1
acuuacuuccaaaacg

SEQ ID NO: 36 - RNA, 39 nt, Artificial Sequence
  chemically treated RNA after digestion with RNase T1
ucaaaacuucucuaaacacauuacuaaaauaacauuucg

SEQ ID NO: 37 - RNA, 17 nt, Artificial Sequence
  chemically treated RNA after digestion with RNase T1
uaucuaaaccuucuacg

SEQ ID NO: 38 - RNA, 11 nt, Artificial Sequence
  chemically treated RNA after digestion with RNase T1
cauacacuacg

SEQ ID NO: 39 - RNA, 17 nt, Artificial Sequence
  chemically treated RNA after digestion with RNase T1
acuacauaaaauuuacg

SEQ ID NO: 40 - RNA, 16 nt, Artificial Sequence
  chemically treated RNA after digestion with RNase T1
auuuuauacccacacg

(19) United States
(12) Patent Application Publication
(10) Pub. No.: US 2009/0075251 A1
(43) Pub. Date: Mar. 19, 2009

(54) Method for analysis of cytosine methylation

(76) Inventors: Dimo Dietrich, Berlin (DE); Philipp Schatz, Berlin (DE); Matthias Schuster, Berlin (DE); Antje Kluth, Wentorf (DE)

Correspondence Address: KRIEGSMAN & KRIEGSMAN, 30 TURNPIKE ROAD, SUITE 9, SOUTHBOROUGH, MA, 01772, US

(21) Appl. No.: 10/594,013

(22) PCT Filed: Mar. 24, 2005
(86) PCT No.: PCT/EP2005/003366

§ 371 Date: Aug. 27, 2008

Related U.S. Application Data
(60) Provisional application No. 60/634,820, filed on Dec. 10, 2004.

(30) Foreign Application Priority Data
Mar. 24, 2004 (EP) 04090117.5
Nov. 12, 2004 (EP) 04090431.0

(51) Int. Cl. C12Q 1/68

(52) U.S. Cl. 435/6

(57) ABSTRACT

A method for the analysis of cytosine methylations in DNA is described. Here, the DNA to be investigated is first chemically or enzymatically converted. Then a promoter is introduced into the DNA. After this, the DNA is converted to RNA. The methylation pattern of the DNA can be concluded in different ways by means of an analysis of the RNA. The RNA is preferably fragmented chemically or enzymatically prior to the analysis, whereby the fragmenting results depend on the methylation pattern of the DNA. The method according to the invention is particularly suitable for the diagnosis and prognosis of cancer disorders and other diseases associated with a modification of the methylation pattern.

The present invention concerns a method for the analysis of methylated cytosine positions in DNA.

### BACKGROUND OF THE INVENTION

5-Methylcytosine is the most frequent covalently modified base in the DNA of eukaryotic cells. It plays an important biological role, among others, in the regulation of transcription, in genetic imprinting and in tumorigenesis (for review: Millar et al.: Five not four: History and significance of the fifth base. In: The Epigenome, S. Beck and A. Olek (eds.), Wiley-VCH Publishers, Weinheim 2003, pp. 3-20). The identification of 5-methylcytosine as a component of genetic information is thus of considerable interest. A detection of methylation is difficult, of course, since cytosine and 5-methylcytosine have the same base-pairing behavior. Many of the conventional detection methods based on hybridization thus cannot distinguish between cytosine and methylcytosine. In addition, methylation information is completely lost in a PCR amplification.

The conventional methods for methylation analysis operate essentially according to two different principles. In the first, methylation-specific restriction enzymes are used, and in the second, a selective chemical conversion of unmethylated cytosines to uracil is employed (so-called: bisulfite treatment, see, e.g.: DE 101 54 317 A1; DE 100 29 915 A1). Since the treatment with methylation-specific restriction enzymes is limited to specific sequences by the sequence specificity of the enzymes, a bisulfite treatment is conducted for most applications (for review: DE 100 29 915 A1, p. 2, lines 35-46). The chemically pretreated DNA is then for the most part amplified and can be analyzed in different ways (for review: WO 02/072880, p. 1 ff; Fraga and Esteller: DNA Methylation: A Profile of Methods and Applications. Biotechniques 33:632-649, September 2002). A selective amplification only of methylated (or in the opposite approach, only of unmethylated) DNA can be conducted with the use of methylation-specific primers or blockers (so-called methylation-sensitive PCR/MSP or the Heavy Methyl method, see: Herman et al.: Methylation-specific PCR: a novel PCR assay for methylation status of CpG islands. Proc Natl Acad Sci USA. 1996 September 3; 93(18):9821-6; Cottrell et al.: A real-time PCR assay for DNA-methylation using methylation-specific blockers. Nucl. Acids. Res. 2004 32: e10). The detection of amplificates is performed by means of different methods, e.g., by gel electrophoresis, chromatography, mass spectrometry, hybridization on oligomer arrays, sequencing, primer extension or real-time PCR variants (see Fraga and Esteller 2002, loc. cit.).

Based on the particular biological and medical importance of cytosine methylation, there is a particular technical need for sensitive, simple, rapid and cost-favorable methods for methylation analysis. Such a method is described in the following. First, a bisulfite conversion of the DNA is carried out. After this, the bisulfited DNA is transcribed into RNA and subsequently the transcripts are further analyzed. The analysis of the transcripts has several technical advantages in comparison to the analysis of the DNA. Thus, the RNA is better suitable for a mass-spectrometric investigation than is DNA (see below). Also, a detection may be simpler to perform by means of hybridization due to the single-strandedness of the RNA (see below). In addition, the RNA—but not the DNA—can be chemically or enzymatically fragmented such that the fragmentation pattern is dependent on the original methylation state of the DNA (see below). Not lastly, the conversion to RNA also permits the application of amplification methods based on transcription. This is associated with several advantages (see below).

In fact, individual ones of the particular embodiments described in the following are already known for the analysis of mutations or polymorphisms. Of course, the present invention, combines, for the first time, a bisulfite treatment with a conversion of the DNA into RNA and a subsequent analysis of the RNA. Thus, access to these already established technologies for nucleic acid analysis is opened up for methylation analysis. Based on the special significance of cytosine methylation and based on the great technical need for powerful methods of methylation analysis, opening up these technologies represents a significant technical advance.

A particular embodiment of the method according to the invention for methylation analysis is characterized in that the bisulfited DNA is converted into RNA by means of an amplification method based on transcription (TAS—transcription based amplification system). NASBA™, 3SR™ or TMA™ are included in these methods. The particulars of these methods are known to the person skilled in the art (for review: Deiman et al., Characteristics and applications of nucleic acid sequence-based amplification (NASBA). Mol Biotechnol 2002 February; 20(2):163-79 with additional citations). When compared with the known PCR methods, the application of the TAS amplification method has several advantages, which are described in detail in the above-cited publications. The isothermal reaction course is particularly included here.

In a particularly preferred embodiment of the TAS method according to the invention, the amplification is conducted in the presence of so-called blocker oligonucleotides. The blockers bind to the so-called “background nucleic acids” and make their amplification difficult. Thus, an increase in the specificity of the methylation analysis can be achieved. Background nucleic acids are understood as those RNAs or DNAs, which bear the same base sequence as the DNA that is to be detected, but are provided, of course, with another methylation state. A frequent problem in methylation analysis, particularly in diagnostic applications, consists of the fact that there is a large amount of background DNA present in the sample material, in addition to the DNA (e.g., disease-specific DNA) that is to be detected. If this background DNA is also detected, this can lead to false-positive results. The use of blocker oligonucleotides according to the invention, in contrast, leads to an increased specificity and to a reduced risk of false-positive results.

The use of methylation-specific blocker oligonucleotides in methylation-specific PCR is already known (so-called HeavyMethyl™ method, Cottrell et al. 2004, loc. cit.). The use of blockers in an isothermal amplification method for methylation analysis, of course, has still not been described.

Another particular embodiment of the method according to the invention for the methylation analysis is characterized in that the bisulfited DNA is converted into RNA, and the RNA is then fragmented chemically or enzymatically in such a way that the fragmentation pattern is dependent on the original methylation state of the DNA. The fragments can be detected, among other ways, by chromatography or mass spectrometry (see below). This method has several advantages when compared with the known methods for methylation analysis. For example, it is possible to clarify detailed methylation patterns within a CpG island in an allele. The current methods for methylation-specific detection, in contrast, are hardly able to simultaneously detect the methylation states of several cytosine positions. Only bisulfite sequencing methods permit the detection of individual cytosine methylations. Bisulfite sequencing, however, has the disadvantage that positions in the direct vicinity of the sequencing primer can only be detected with difficulty. The same applies to positions which are far removed from the start of sequencing. In addition, the method according to the invention is faster, more cost-favorable and easier to automate than sequencing.

In another particular embodiment of the method according to the invention, the transcripts are analyzed by mass spectroscopy. RNA is better suitable for a mass-spectrometric investigation than is DNA. Here, the 2′-OH group of the ribose ring stabilizes the N-glycosidic bond between nucleobase and ribose. The depurination typical in a mass-spectrometric analysis is thus prevented. In this way, RNA is better suitable for this type of analysis than is DNA (see: Kirpekar et al.: Matrix assisted laser desorption/ionization mass spectrometry of enzymatically synthesized RNA up to 150 kDa. Nucl. Acids. Res. 1994 22: 3866-3870; Nordhoff et al.: Ion stability of nucleic acids in infrared matrix-assisted laser desorption/ionization mass spectrometry; Nucl. Acids. Res. 1993, 21: 3347-3357).

A particularly preferred embodiment included in the embodiments of the method according to the invention combines a methylation-specific enzymatic fragmenting (see above) with a subsequent mass-spectrometric analysis. Thus, RNA is preferably fragmented by means of the enzyme RNase-T1 and then is analyzed by means of MALDI. Similar methods for the detection of single nucleotide polymorphisms (SNPs) or short tandem repeats (STRs) have already been described. (Krebs et al.: RNaseCut: a MALDI mass spectrometry-based method for SNP discovery. Nucleic Acids Res. 2003 April 1; 31 (7):e37.; Seichter et al.: Rapid and accurate characterisation of short tandem repeats by MALDI-TOF analysis of endonuclease cleaved RNA transcripts. Nucleic Acids Res. 2004 January 20; 32(2):E16.; Hartmer et al.: RNase-T1 mediated base-specific cleavage and MALDT-TOF MS for high-throughput comparative sequence analysis. Nucleic Acids Res. 2003 May 1; 31 (9): e47). In the case of SNP or STR analysis, transcription and fragmenting are conducted, of course, only in order to facilitate a mass-spectrometric analysis of the DNA. In this case, the number of enzyme cleavage sites remains the same and the short RNA fragments that form are distinguished only on the basis of base composition. Thus, the differences in mass of the fragments can be very small (approximately 1-40 Da in the case of SNPs). A conclusion in regard to the fragmentation pattern at the positions to be investigated is not possible according to the already-described method. The application of the already-known methodology to methylation analysis thus leads to unexpected advantages, since here the number of enzyme cleavage sites correlates directly with the methylation of the DNA to be investigated.

### DESCRIPTION

The invention involves a method for the analysis of cytosine methylations in DNA, in which the following steps are conducted:

1) the DNA to be investigated is reacted so that 5-methylcytosine remains unchanged, while unmethylated cytosine is converted to uracil or to another base which differs from cytosine in its base-pairing behavior,
2) a promoter sequence is introduced into the DNA,
3) RNA is transcribed,
4) the RNA is analyzed, [and]
5) a conclusion with regard to the methylation state of the investigated DNA is made.

In the first step of the method according to the invention, the DNA to be investigated is reacted with a chemical or with an enzyme so that 5-methylcytosine remains unchanged, while unmethylated cytosine is converted to uracil or to another base which differs from cytosine in its base-pairing behavior. The DNA to be investigated thus can originate from different sources depending on the diagnostic or scientific objective. For diagnostic objectives, tissue samples are preferably used as the initial material, but body fluids, particularly serum, can also be used. It is also possible to use DNA from sputum, stool, urine, or cerebrospinal fluid. Preferably, the DNA is first isolated from the biological specimen. The DNA is extracted according to standard methods, from blood, e.g., with the use of the Qiagen UltraSens DNA extraction kit. The isolated DNA can then be fragmented, e.g., by reaction with restriction enzymes. The reaction conditions and the enzymes that can be employed are known to the person skilled in the art and result, e.g., from the protocols supplied by the manufacturers. Then the DNA is chemically or enzymatically converted. A chemical conversion by means of bisulfite is preferred. The bisulfite conversion is known to the person skilled in the art in different variations (see, e.g.: Frommer et al.: A genomic sequencing protocol that yields a positive display of 5-methylcytosine residues in individual DNA strands. Proc Natl Acad Sci USA. 1992 March 1; 89(5): 1827-31; Olek, A modified and improved method for bisulphite based cytosine methylation analysis. Nucleic Acids Res. 1996 December 15; 24(24): 5064-6.; DE 100 29 915; DE 100 29 915). The bisulfite conversion is most preferably conducted in the presence of denaturing solvents, e.g., dioxane, and a radical trap (see: DE 100 29 915). In another preferred embodiment, the DNA is not chemically converted, but rather enzymatically converted. This is conceivable, e.g., with the use of cytidine deaminases; unmethylated cytidines react more rapidly than methylated cytidines. A corresponding enzyme has been recently identified (Bransteitter et al.: Activation-induced cytidine deaminase deaminates deoxycytidine on single-stranded DNA but requires the action of RNase. Proc Natl. Acad Sci USA. 2003 April 1; 100(7): 4102-7).

In the second step of the method according to the invention, a promoter, which makes possible a conversion of the DNA to be investigated into RNA, is introduced into the pretreated DNA. Various methods are known to the person skilled in the art for this purpose. In a preferred embodiment of the invention, a PCR is carried out, in which one of the primers bears a promoter sequence. In another preferred embodiment, the NASBA method or another amplification method based on transcription is used, in which RNA amplificates can be produced starting from DNA (see the details below). It is, however, also conceivable to use other amplification methods, e.g., the rolling circle method. The amplification is preferably conducted in a manner that is not methylation-specific. It is, however, also possible to amplify a larger sequence region in a methylation-specific manner and to analyze specific cytosine positions within this sequence by means of the method according to the invention. The combination of methylation-specific amplification and RNA transcription makes it possible to first propagate the methylated subpopulation in the primer binding sequence from a mixture of different DNAs and to investigate this subpopulation more precisely for its methylation. In this way, special methylation patterns can be investigated more precisely, e.g., for the investigation of sequences which are methylated at their 5′ end and unmethylated at their 3′ end. These sequences are particularly interesting for demonstrating DNA methylation.

In addition, it is conceivable to ligate the promoter sequences independently from an amplification of the DNA. This is possible, e.g., if the bisulfite DNA is cloned into a vector which already bears a promoter. A ligation without prior amplification then has the advantage that the quantity of RNA, which is produced later by the transcription, is linearly related to the DNA that is used. In contrast, the PCR-based methods lead to an exponential amplification, which could make a quantification difficult.

Preferably, T7, T3 or SP6 sequences are used as promoters. However, other RNA polymerase promoters may also be used. Promoter sequences are known to the person skilled in the art.

The transcription is conducted in the third step of the method according to the invention. The RNA polymerases necessary for this are aligned along the incorporated promoter sequences. The transcription conditions are dependent on the polymerases that are utilized. The details are known to the person skilled in the art.

In the fourth step of the method according to the invention, the transcripts are analyzed. The original methylation state of the investigated DNA can be concluded from the results, in the fifth step. The analysis of the transcripts can be performed by a plurality of known molecular-biological methods, e.g, via hybridization or sequencing. In a preferred embodiment, detection is made via a hybridization on a microarray. A microarray-based detection can be simpler with transcripts than with DNA, since the RNA is already present in single-stranded form and thus no longer needs to be denatured prior to the hybridization. Measures that prevent a decomposition of the RNA are known to the person skilled in the art. For hybridization to an array, the RNA is provided beforehand with a label, preferably a fluorescent label. This can be done, e.g., with the help of a transcription kit, in which nucleotides labeled with AminoAllyl are incorporated in the RNA (Amino Allyl MessageAmp™ Kit; Ambion, USA). The AminoAllyl nucleotides are used by the RNA polymerases with an efficiency that is nearly equal to that of natural nucleotides. After the transcription, a dye is coupled to the modified nucleotides. Additional methods for labeling RNAs are part of the prior art (see, e.g.: Monnot et al.: Labeling during cleavage (LDC), a new labeling approach for RNA. Nucleosides Nucleotides Nucleic Acids. 2001 April-July; 20(4-7): 1177-9. Proudniko and Mirzabekov: Chemical methods of DNA and RNA fluorescent labeling. Nucleic Acids Res. 1996 November 15; 24(22): 4535-42).

In another preferred embodiment of the method according to the invention, the RNA is analyzed by a mass-spectrometric method, e.g., via electrospray or PSD mass spectrometry (see: Little et al.: Verification of 50- to 100-mer DNA and RNA sequences with high-resolution mass spectrometry. Proc Natl Acad Sci USA. 1995 March 14; 92(6): 2318-22). The use of RNA here instead of DNA has the advantage that the RNA is more stable during the mass-spectrometric analysis and provides better flight properties than does DNA. In another preferred embodiment of the method according to the invention, the RNA is analyzed by means of an RNA protection assay. The details are known to the person skilled in the art. Other analytical methods are conceivable, which utilize the single-strandedness of the RNA or its particular chemical or physical properties and thus are more advantageous than a direct detection of the DNA. The use of these methods is also a part of this invention.

In a preferred embodiment of the invention, the RNA is chemically or enzymatically fragmented prior to the analysis. In this way, the mass-spectrometric analysis can be particularly facilitated (see: Krebs et al. 2003, loc. cit.; Seichter et al. 2004, loc. cit.; Hartmer et al. 2003, loc. cit.).

### Particularly Preferred Embodiments

### Application to Transcription-Based Amplification Methods

In a particularly preferred embodiment of the method according to the invention, the introduction of the promoter sequence and the transcription are conducted in parallel by means of an amplification method based on transcription. Correspondingly, this embodiment can be described as follows:

The method for the analysis of cytosine methylations in DNA is characterized in that the following steps are conducted:

1) the DNA to be investigated is reacted so that 5-methylcytosine remains unchanged, while unmethylated cytosine is converted to uracil or to another base which differs from cytosine in its base-pairing behavior,
2) the converted DNA is amplified by means of an amplification method based on transcription,
3) the amplificates are analyzed, [and]
4) a conclusion is made with regard to the methylation state of the investigated DNA.

As initial material for the method according to the invention, the specimens described more precisely above can serve for this purpose. The bisulfite conversion is performed also as presented above. In the second step of this embodiment, the converted DNA is amplified by means of an amplification method based on transcription, particularly by means of NASBA™, 3SR™ or TMA™. These methods are known in detail to the person skilled in the art (see, e.g., Deiman et. al 2002, loc. cit.). An application of these methods to the investigation of cytosine methylations, of course—insofar as this can be ascertained—has still not been described.

The amplification methods based on transcription imitate retroviral replication. The amplification of the target sequence is usually performed by means of two primers and three enzymes. A T7 promoter sequence, by means of which RNA can then be generated by means of a T7 polymerase, is introduced into the target sequence via one of the primers. The RNA is again converted into DNA by means of a reverse transcriptase and RNA-DNA intermediates that have formed in the meantime are decomposed by means of an RNase-H. The amplification is performed isothermally, usually at 41° C. The generated amplificates can be detected by means of a plurality of different methods, e.g., via gel electrophoresis, diverse chromatographic methods or the use of labeled, particularly fluorescently labeled, probes. Also, the use of real-time probes (Molecular Beacon) has been described in the meantime (see: Deiman et al. 2002, loc. cit.). In a preferred embodiment, the detection is made by methylation-specific probes, which bind specifically only to amplificates with a specific methylation state.

It is known to the person skilled in the art how to conduct the above-described method. In particular, he knows the reaction conditions, the reaction components, the design of the primers and the analytical methods (for review, see: Deiman et al., 2002, loc. cit.).

According to the invention, amplification methods based on transcription are applied in order to specifically detect the DNA of a certain methylation state. This is possible, on the one hand, via a methylation-specific amplification by means of methylation-specific primers or methylation-specific blocker oligonucleotides (see below for details of the blockers). In addition to this, it is also conceivable to amplify the DNA in a way that is not methylation-specific, but to detect the amplificates by means of methylation-specific probes. It is also possible to combine methylation-specific amplification and methylation-specific detection.

The principle of the use and of the design of methylation-specific primers is known to the person skilled in the art, particularly from the so-called “MSP method” (methylation-specific PCR) (see: Herman et al., 1996, loc. cit.). Methylation-specific primers preferably bind only to that DNA which has the methylation state that is to be detected. Correspondingly, the methylation-specific primers bear at least one CpG dinucleotide (for the detection of methylated DNA) or a methylation-specific TG or CA dinucleotide (for the detection of unmethylated DNA on the two possible DNA strands). The principles for the design of methylation-specific primers are known to the person skilled in the art: The higher the number of methylation-specific dinucleotides and the shorter the length of the primers, the greater will be the specificity of the amplification. On the other hand, the application range of the method will be more greatly limited due to the sequence requirements, the greater the number of methylation-specific dinucleotides contained in the primers. As a rule, 1 to 4 methylation-specific dinucleotides will be used for MSP primers.

The criteria for primer design known from MSP are valid in principle also for the method according to the invention. Here, of course, it should be considered that the amplification is conducted isothermally at only 41° C. Therefore, the primers must contain more methylation-specific dinucleotides, in comparison to MSP, in order to attain a comparable specificity.

In principle, it is sufficient according to the invention, if only one of the two primers is constructed in a methylation-specific manner. It is preferable, however, if both primers are methylation-specific.

### Particularly Preferred Embodiments

### Use of Amplification Methods Based on Transcription in Combination with Methylation-Specific Blocker Molecules

As has already been described above, particularly for diagnostic applications, there is a great technical need for methods which can specifically detect methylation patterns, when a high background of DNA of the same sequence but of a different methylation pattern is present in the specimen along with the DNA to be detected. The danger exists here, in particular, of false-positive results. One possibility for increasing the specificity of the amplification is the use of methylation-specific blocker molecules. These blockers bind specifically to the background DNA and thus prevent their amplification. This use of blockers in a methylation-specific PCR has already been described several times (so-called HeavyMethyl™ method, PCT/EP02/02527). The use of methylation-specific blockers has several advantages in comparison to the use of methylation-specific primers (see: Cottrell et al. 2004).

The following particular embodiment of the method of the invention combines, for the first time, the amplification of bisulfited DNA by means of an amplification method based on transcription with the use of methylation-specific blockers. This embodiment can be described as follows:

The method for the analysis of cytosine methylations in DNA is characterized in that the following steps are conducted:

1) the DNA to be investigated is reacted so that 5-methylcytosine remains unchanged, while unmethylated cytosine is converted to uracil or to another base which differs from cytosine in its base-pairing behavior,
2) the converted DNA is amplified by means of an amplification method based on transcription, wherein the amplification occurs in the presence of at least one methylation-specific blocker molecule, which binds specifically to the background nucleic acid and hinders the amplification thereof,
3) the amplificates are analyzed, [and]
4) the methylation state of the investigated DNA is concluded.

“Background nucleic acid” here is understood to be a nucleic acid which can be attributed to a DNA which has the same sequence, but provides a methylation state that is different from the DNA to be detected. Since the amplification based on transcription takes place predominantly via RNA intermediates, methylation-specific blockers can bind to the background RNA and hinder the amplification thereof. Nevertheless, the amplification cycle also takes place via a primer extension. This step would block the background DNA when the blocker binds to the background DNA. In the optimal case, the blocker blocks the amplification both via the RNA as well as also via the DNA.

In principle, the blocker technology known from the “HeavyMethyl™” method is applicable to the above-described embodiment. This is described in detail in the WO Application PCT/EP02/02572, which is expressly referenced here. The blockers preferably involve oligonucleotides, but they may also involve other molecules, particularly PNAs. In the method according to the invention, RNA blockers may also be utilized, since RNA-RNA* hybrids are particularly stable. The blockers are methylation-specific, i.e., they bear at least one CpG dinucleotide or a methylation-specific TG or CA dinucleotide (see above relative to the primers). The primers are preferably added in excess to the reaction batch. In particular embodiments, two or more blockers are used, which preferably overlap with the binding sites of the primers, in order to thus additionally prevent an amplification. In addition, the blockers may be chemically modified so that an extension or a decomposition of the blockers does not occur due to the polymerase in the course of the amplification (see for details: PCT/EP02/02572; Cottrell et al. 2004, loc. cit.). It is known to the person skilled in the art that all known embodiments of blocker technology, and particularly those described in the above-cited publications, can also be extensively applied to the combination of amplification methods based on transcription and the use of blockers according to the invention. The corresponding embodiments are thus also part of this invention.

In fact, the use of blockers in methylation-specific PCR is already known, but the method according to the invention offers a decisive advantage in comparison to the already described methods. The blocker oligonucleotides bind to the background RNA and thus form RNA-DNA hybrids. The RNA part of these hybrids can be broken down by the RNase-H enzyme in the reaction cycle and thus can be removed from the entire amplification reaction.

The use of blockers here thus leads not only to a blocking of the amplification of the background nucleic acid, as in the case of the known Heavy-Methyl™ method, but, in addition to this, to a decomposition of the background nucleic acid. An increased specificity of the reaction results from this.

In this embodiment of the method according to the invention, the amplification can take place both by means of methylation-specific primers (see above) as well as also by means of primers that are not methylation-specific. In a preferred embodiment, primers that are not methylation-specific are utilized.

The amplification is produced in the presence of at least one methylation-specific blocker oligomer. These blockers bear correspondingly at least one CpG position or a methylation-specific TG or CA position. The oligomers preferably bear 3-5 methylation-specific positions. Oligonucleotides are preferably used, since the corresponding hybrids of blocker and RNA can be recognized particularly effectively by the RNase-H. The blocker oligonucleotides are preferably between 10 and 25 nucleotides long. The blockers are added in excess to the primers in the reaction batch, particularly preferably in a 3 to 15-fold higher concentration.

The blockers can be chemically modified at the 3′ and/or 5′ end, in order to prevent an extension or a decomposition of the blockers. The details for this are known to the person skilled in the art (PCT/EP02/02572).

The amplification then occurs under the above-described conditions. An NASBA reaction is preferably conducted. Correspondingly, one of the primers bears a T7 promoter, which serves as the starting point of transcription for the RNA polymerase. The primer hybridizes to the (+)-strand of the target sequence. As a rule, a short heating step is provided for this purpose. The primer is extended by the reverse transcriptase with the formation of a DNA double strand. After another heating step, the second primer can bind to the likewise generated (−)-DNA strand. A DNA double strand, which bears a complete T7 promoter, will then be formed by another primer extension. The binding of the methylation-specific blockers to the background DNA here blocks an extension of the background DNA. Following this, transcripts, which will again be converted into DNA double strands via RNA-DNA hybrids, are generated from the T7 promoter. In this way, the methylation-specific blocker oligonucleotides in turn bind to the background DNA and thus prevent its amplification. The RNA part of the formed blocker-RNA hybrids will thus be decomposed by the RNase-H. The background RNA is thus no longer available as a template for further rounds of amplification. The amplification of the DNA/RNA to be detected, in contrast, is not adversely affected by the blockers.

In a particularly preferred embodiment of the method according to the invention, the amplificates are detected by means of real-time probes. A real-time detection of NASBA amplificates by means of Molecular Beacons has already been described (Deiman et al. 2002, loc. cit). However, the use of other real-time probes is also conceivable, particularly the application of Lightcycler™ probes. These probes are preferably methylation-specific, i.e., they bear at least one methylation-specific dinucleotide (see above). Details for the construction of corresponding probes are known to the person skilled in the art (see: PCT/EP02/02572; U.S. Pat. No. 6,331,393).

The particularly preferred embodiment of the method according to the invention with the use of methylation-specific blocker oligonucleotides is shown in Table 1. A comparison to the already known NASBA methods is also found therein.

### Particularly Preferred Embodiments

### Analysis by Means of Fragmenting the RNA

In another particularly preferred embodiment of the method according to the invention, the RNA is chemically or enzymatically fragmented prior to the analysis. In this way, the mass-spectrometric analysis can be particularly facilitated (see: Krebs et al. 2003, loc. cit.; Seichter et al. 2004, loc. cit.; Hartmer et al. 2003, loc. cit.).

In a particularly preferred embodiment of the method according to the invention, the RNA is fragmented as a function of the methylation state prior to the analysis. The methylation pattern can then be concluded from the fragmentation pattern. The basis for the possibility of a methylation-dependent fragmenting is the bisulfite conversion (or an analogous chemical or enzymatic conversion) in combination with an amplification. It is possible in this way to generate nucleic acids which bear cytosines or guanines precisely and only at those sites where a methylcytosine existed in the original DNA. The nucleic acids are then specifically cleaved at the C or G positions. Specific fragmentation patterns then result for the original methylation state, and these patterns can be analyzed by different methods.

In the bisulfite conversion, first all cytosines are converted to uracil, while methylated cytosines remain unchanged. Two DNA strands are thus formed, which are no longer complementary to one another. After an amplification, of course, there are again two complementary DNA strands. One of the strands contains cytosines only at those sites where methylcytosines existed in the original DNA. This strand is denoted in the following as G-rich, since it is comparatively poor in cytosines. If a promoter sequence had been introduced into this G-rich strand, then a complementary-now C-rich-RNA molecule can be transcribed. In this C-rich molecule, guanines are represented only at those sites where methylcytosines existed in the original DNA. The guanines in this RNA transcript thus exactly illustrate the methylation state of the original DNA. Correspondingly, an RNA molecule can be generated, in which all cytosines reflect a methylcytosine. The guanine or cytosine positions can then be specifically cleaved. Both enzymatic as well as chemical methods are conceivable for this purpose. For the specific enzymatic cleavage at G positions, the enzyme RNase-T1 is particularly preferably used (see: Hartmer et al. 2003, loc. cit.; Krebs et al. 2003, loc.cit.). The enzyme is commercially available from different manufacturers (e.g., Roche Diagnostics, Mannheim, Germany). A specific cleavage of RNA at C positions is possible, e.g., by means of RNase-A, as long as chemically modified uracil ribonucleotides are utilized in the transcription (see: Krebs et al. 2003, loc. cit.). A specific chemical cleavage at C or G positions is possible by means of different reagents (see: Peattie: Direct chemical method for sequencing RNA. Proc Natl Acad Sci USA. 1979 April; 76(4): 1760-49): Krebs et al. 2003, loc.cit.).

Specific fragmentation patterns which correspond to the local distribution of methylcytosines on the original DNA to be investigated result due to the cleavages. Each fragment that is formed thus represents the region between two methylated cytosines in the original DNA. The number of fragments that are formed correlates directly with the number of methylated cytosines. The property that only the originally methylated positions are the starting point for a fragmentation represents a decisive feature of this particularly preferred embodiment. In the known methods for mutation/polymorphism analysis, the number of fragmentation sites is independent of the sequence of the initial specimen. Following a fragmenting, there is always formed the same number of fragments, which do not differ in the number of nucleotides, but rather only in the base composition. As a rule, this leads to rather small chemical-physical differences in the fragments, which can no longer be resolved under certain circumstances during the analysis. Additionally, this fragmenting sites that are not sequence-specific is characterized in that there is a tendency for a great many fragments to form (statistically, every fourth nucleotide is cleaved), which thus are very small and are difficult to analyze. These small fragments are particularly indistinguishable in a chromatographic analysis.

The methods described here overcome these disadvantages. Over and above this, they contain another decisive advantage. Since the fragmenting results only at originally methylated sites, each fragment that forms represents the sequence in between the adjacent methylated cytosines. If, for example, unmethylated CpG sites are found in between, then, for a single initial DNA molecule, combined information can be obtained via the methylation of these CpGs. Furthermore, a fragmenting at an originally methylated site also influences the adjacent fragment, since, obviously, two adjacent fragments provide information on the same CpG site. Therefore, this adjacent fragment and the methylation state reflected therewith can also be assigned to a single initial DNA molecule. In this way, e.g., genetic imprinting, which occurs in an allele-specific manner, or the activity of methyltransferases can be investigated more precisely. This clear assignment of the methylation state to a single initial molecule cannot be achieved with fragmenting that is not methylation-specific. This is of interest precisely in the case of DNA mixtures, which, as a rule, contain a complex mixture of different methylated DNA molecules, of which, for the most part, only subpopulations are of interest.

In comparison to other fragmenting-based methods, in the case of the methods described here, a somewhat less complex fragmenting occurs, since cleavage occurs only at originally methylated CpG sites. But conversely, this makes possible the analysis of rather complex analytes. Thus, for example, several different loci in the genome can be investigated simultaneously in the same reaction. This method can therefore be multiplexed, which is a decisive advantage, if only a limited quantity of initial specimen material is available. Other fragmenting methods generate a large quantity of small fragments, and these can no longer be assigned to individual loci in a multiplex reaction.

The methylation state of all cytosines contained in the DNA amplificate can be determined via a suitable analysis of the fragments that form (see FIG. 1). Different methods are available for this. In a preferred embodiment, mass-spectrometric methods, particularly MALDI-TOF, are utilized. By the precise mass of the fragments and the knowledge of the sequence of the initial DNA, it can thus be determined exactly which two cytosines—namely those delimiting the fragment—were methylated. The details of MALDI-TOF analysis are known to the person skilled in the art. In particular, in US Patent Application US 2003 0129589, a plurality of possibilities for mass-spectrometric analysis is given, which in many cases are correspondingly applicable to the method according to the invention. In other preferred embodiments, the analysis of the fragmentation pattern of the RNA is performed via electrophoretic or chromatographic methods (e.g., capillary gel electrophoresis or HPLC). These methods make possible a quantification of the RNA fragments that form by integration of the signal intensities (this is known to the person skilled in the art). If the DNA to be investigated is present as a mixture of different methylated species, then a conclusion relating to the mixing ratio that is present for this species can be achieved by such quantification.

Other fragmenting-based methods are only suitable within certain limits for such electrophoretic and chromatographic analysis methods, since in the case of fragmenting that is not methylation-specific, only the base composition and not the number of bases is distinguished in a fragment. This base number cannot be resolved, e.g., with capillary gel electrophoresis. This represents another advantage of the described methods.

In a particularly preferred embodiment of the method of the invention, in addition to the promoter, control sequences are also introduced into the DNA, and these form the basis for being able to examine the completeness of the fragmenting. For example, if the G-rich primer bears the control sequence “TCTTTTC”, then an RNA with the additional sequence GAAAAGA results. All other guanines in this RNA originate from methylated cytosines in the original DNA. The completeness of the fragmenting reaction can be monitored via detection of the control sequence fragments (see: Examples; FIG. 2).

### Use of the Method According to the Invention

The above-described methods are particularly preferably used for the diagnosis or prognosis of cancer disorders or other diseases associated with a change in the methylation state. These include, among others, CNS malfunctions; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disease of the skin, the muscles, the connective tissue or the bones; endocrine and metabolic malfunction, damage or disease; headaches or sexual dysfunction. The method according to the invention is also suitable for predicting undesired drug effects and for distinguishing cell types or tissues or for investigating cell differentiation.

### Kits According to the Invention

The following kits are also [provided] according to the invention:

A kit, which consists of a bisulfite reagent and of at least one primer, which bears a promoter.

Said kit, which additionally contains enzymes and/or other components for conducting an amplification method based on transcription.

Said kit, which additionally contains at least one methylation-specific blocker oligomer.

A kit, which consists of a bisulfite reagent, primers and an enzyme that cleaves RNA in a nucleotide-specific manner, and, optionally, a polymerase and other reagents necessary for an amplification.

### EXAMPLES

### Example 1

### Investigation of the Promoter Region of the Human Adenomatosis Polyposis Coli (APC) Gene

The methylation state of the promoter region of the human adenomatosis polyposis coli (APC) gene (NM_{—}000038.2) will be investigated. Here a DNA was used, which was methylated synthetically by an enzyme which methylates all cytosines in the CpG-context (Sssl methyltransferase). After a bisulfite treatment of the DNA, a region of the promoter was amplified by means of a PCR. The following conditions were selected for the PCR: 1 U (0.2 μl) of HotStarTaq polymerase (Qiagen), 0.2 μl of dNTP mix (25 mmol/l each of dATP, dGTP, dCTP and dTTP, Fermentas), 2.5 μl of 10×PCR buffer (Qiagen), 2 μl of primer mix (6.25 μmol/l of each, MWG Biotech AG), 1 μl of partially deaminated DNA (10 ng), 19.1 μl of water; Temperature program: 10 min at 95° C., and subsequently 40 cycles with 30 sec at 95° C., 45 sec at 55° C. and 1:30 min at 72° C. The following two primers were used for this amplification: TCTTTTCGGTTAGGGTTAGGTAGGTTGT (G-rich) (Seq ID 1) and GTAATACGACTCACTATAGGGAGACTACACCAATACMCCACATATC (C-rich) (Seq ID 2). The underscored part of the C-rich primer thus represents the promoter for the T7 polymerase. In the G-rich primer there is still contained an additional sequence (underscored), which, after the transcription of the PCR product, is localized in a reverse complementary manner in an RNA molecule at the 3′ end of this product and thus, after cleavage by the RNase-T1, emits a signal indicating that transcription is complete. This sequence thus represents a control fragment after the endonuclease treatment, which is always formed independently of the methylation state. The following conditions were selected for the transcription of the PCR product: 10 μl of PCR product, 5 μl of 5× T7 RNA polymerase buffer (Fermentas), 1 μl of T7 polymerase (20 U/μl, Fermentas), 0.5 μl of NTP mix (Fermentas, each of 25 mmol/l), 8.5 μl of water. The incubation was conducted for 1.5 h at 37° C. Then the RNase digestion was carried out by adding 2.5 μl of RNase-T1 (10 U/μl, Fermentas) with a 45-minute incubation at 37° C. This reaction batch was then incubated with approximately 20 mg of “clean resins” of the Sequenom company, in order to reduce the Na+ and K+ ion concentration of the solution. Finally, 0.5 μl of the mix containing 0.5 μl of 3-hydroxypicolinic acid was mixed in and examined with a Bruker Reflex 2 MALDI-TOF mass spectrometer in the negative ion mode. In this case, the reflector mode was used.

The transcription of the PCR product results in a product of the following sequence (Seq ID 3):

GGGAGACUACACCMUACAACCACAUAUCGAUCACGUACGCCCACACCCAACCAA UCGACGAACUCCCGACGAAAAUAAAACGCCCUAAUCCGCAUCCAACGAAUUAC ACAACUACUUCUCUCUCCGCUUCCCGACCCGCACUCCGCMUAAAACACAAAACC CCGCCCMCCGCACAACCUACCUMCCCUMCCGAAAAGA. The “GGGAG” sequence at the beginning of this molecule here represents the promoter of the T7 polymerase which was used and which was partially co-transcribed. The “GAAAAGA” sequence at the end of the RNA molecule results from the control sequence additionally appended to the G-rich primer. All other guanines in this molecule resulted from methylated cytosines in the original DNA. If this DNA had not been methylated at these sites, adenines would be found instead of guanines. The RNase-T1 now cleaves the RNA after the guanine and produces a fragmentation pattern that reflects the methylation state of the original DNA. The fragments that form are listed with their corresponding m/z values in Table 2.

TABLE 2

Fragments and their m/z values of the RNA
after a digestion of the APC-198 transcript
with RNase-T1.

| | | |
|---|---|---|
| Fragment No. | Sequence | m/z |

| | | |
|---|---|---|
| 1 | Gp | 345 |
| 2 | Gp | 345 |
| 3 | Gp | 345 |
| 4 | AGp | 674 |
| 5 | ACUACACCAAUACAACCACAUAUCGp | 7938 |
| 6 | AUCACGp | 1920 |
| 7 | UACGp | 1286 |
| 8 | CCCACACCCAACCAAUCGp | 5678 |
| 9 | ACGp | 980 |
| 10 | AACUCCCGp | 2531 |
| 11 | ACGp | 980 |
| 12 | AAAAUAAAAAACGp | 4249 |
| 13 | CCCUAAUCCGp | 3142 |
| 14 | CAUCCAACGp | 2860 |
| 15 | AAUUACACAACUACUUCUCUCUCCGp | 7845 |
| 16 | CUUCCCGp | 2178 |
| 17 | ACCCGp | 1590 |
| 18 | CACUCCGp | 2201 |
| 19 | CAAUAAAACACAAAACCCCGp | 6409 |
| 20 | CCCAACCGp | 2530 |
| 21 | CACAACCUACCUAACCCUAACCGp | 7254 |
| 22 | AAAAGp | 1662 |
| 23 | A | 267 |

The fragments which resulted from the RNase-T1 digestion of the transcript and were detected by means of Maldi-TOF mass spectrometry are shown in FIG. 3. It can be recognized therein that almost all fragments that are characteristic of the completely methylated DNA according to Table 2 could be detected. Only fragments that are smaller than m/z 980 could not be detected, since in this region, the matrix used for the Maldi-TOF analysis generates too high a background signal. It could now be clearly demonstrated by means of this spectrum that the original DNA was methylated at all cytosines in the CpG context.

### Example 2

### Investigation of the Methylation State of the CDH13 Gene

The methylation state of the CDH13 gene will be investigated. For this purpose, Sssl-methylated DNA, unmethylated Phi-DNA and a cloned methylated PCR amplificate were investigated. A sequencing was conducted for the control. The method according to the invention was applied as described above. The following sequences were used as primers:

(Seq ID 4)

TCTTTTTCTTTGTATTAGGTTGGAAGTGGT;

(Seq ID 5)

GTAATACGACTCACTATAGGGAGCCCAAATAAATCAACAACAACA.

The transcription of the amplificates produced the following products:

Methylated DNA:

(Seq ID 6)

GGGAGCCCAAAUAAAUCAACAACAACAUCACGAAAACAUUAAAUAAAAAC
UAAUAACCAAA.ACCAAUAACUUUACAAAACGAAUUCCUUCCUAACGCUC
CCUCGUUUUACAUAACAAAUACGAAAUAAACACCUCGCGAAAAACGAACC
CCGCGAAAAUAACAUCCCAUUUAGUUCUUUAAAĆUAUUAAAACUCAACC
U-ACAAAUCACGCUAAACAAUACCAACUAAUUCCACUUUUCCAAAAAAUA
APAAUUACACGAAAAACUAACGACCACUUCCAACCUAAUACAAAGAAAAA
GA;
Methylated clone:

(Seq ID 7)

GGGAGCCCAAAUAAAUCAACAACAACAUCACAAAAACAUUAAAUAAAAAC
UAAUA.ACCAAAACAAUAÄCUUUACAAAACGAAUUCCUUCCUAACGCUCC
GUCGUUUUACAUAACAAAUACGAAAUAAACACCUCGCGAAAAACGAACCC
CGCGAAAAUAACAUCCCAUUUACUUCUUUAAACUAUUAAAACUCAACCUC
ACAAAUCACGCUAAACAAUACCAACUAAUUCCACUUUUCCAGAAAAUAAA
AUUACACGAAAAACUGACGACCACUUCCAACCUAAUACAAAGAAAAAGA.

The fragments that form are listed with their corresponding m/z values in Table 3.

FIG. 4 shows the fragments which resulted from the RNase-T1 digestion of the transcript and were detected by means of Maldi-TOF mass spectrometry. In this way, for synthetically methylated DNA, all fragments could be detected, which are characteristic of completely methylated DNA (Table 3, columns 1 and 2); only fragments smaller than 980 (m/z) and larger than 15250 (m/z) could not be detected due to device limitations. In Table 3 (columns 3 and 4), the fragmenting of the cloned DNA is additionally shown. In this way, the differences relative to the synthetically methylated DNA, which are described in the following, are visible. The 8619.3 (m/z) fragment is no longer detectable. This is based on the fact that the cytosine, which would lead to the formation of the 8619.3 (m/z) fragment and of the 15723.7 (m/z) fragment in the methylated state of the DNA to be investigated, was obviously not methylated. Therefore, a 24021.8 (m/z) fragment, which corresponds to the combination of these two fragments, is formed. This fragment, however, could not be detected because of its size and the device limitations. In the case of the cloned DNA, two fragments can still be detected with the 10103.1 (m/z) fragment and the 5166.2 (m/z) fragment, which were not at first expected. Their formation results from a conversion of a cytosine outside of the CpG context, which did not take place during the bisulfite treatment of the DNA. An additional cleavage site, which brings about these two fragments, thus had the expected 15253.3 (m/z) fragment. The presence of the 2602.6 (m/z) fragment in the cloned DNA instead of the 3566.2 (m/z) fragment which was expected also has the same cause. Here also, a cytosine had been deaminated outside the CpG context and not in the bisulfite treatment and resulted in a cleavage of the 3566.2 (m/z) fragment into a 2602.6 (m/z) fragment and a 979.6 (m/z) fragment (undetectable). The spectrum of the unmethylated DNA is shown in addition in FIG. 4. As is to be expected, no other detectable fragments occur here in addition to the 1991.3 (m/z) fragment, since the RNA transcript of the unmethylated, bisulfited DNA has no cleavage sites other than those of the already described control sequence at the end of the transcript. All of these interpretations could be confirmed by a sequencing (data not shown).

TABLE 3

Fragments and their m/z values of the RNA after a
digestion of the CDH13 transcript with RNase-T1.

| | Sequence of the RNA fragment | | |
|---|---|---|---|
| m/z | Methylated DNA | Clone | m/z |

| | | | |
|---|---|---|---|
| 8619.3 | CCCAAAUAAAUCAA- | CCCAAAUAAAUCAACAA- | 24021.8 |
| | CCAACAACAUCACGp | CAACAUCACAAAAACAUU- | |
| 15723.7 | AAAACAUUAAAUAA- | AAAUAAAAACUAAUAAC- | |
| | AAACUAAUAACCAA- | CAAAACAAUAACUUUACA- | |
| | AACCAAUAACUUUA- | AAACGp | |
| | CAAAACGp | | |
| 4718.8 | AAUUUCCUUCCU- | AAUUCCUUCCUAACGp | 4718.8 |
| | AACGp | | |
| 2483.5 | CUCCCUCGp | CUCCCUCGp | 2483.5 |
| 5731.4 | UUUUACAUAACAA- | UUUUACAUAACAAAUACGp | 5731.4 |
| | AUACGp | | |
| 4482.7 | AAAUAAACACCUCGp | AAAUAAACACCUCGp | 4482.7 |
| 650.4 | CGp | CGp | 650.4 |
| 2296.4 | AAAAACGp | AAAAACGp | 2296.4 |
| 2224.3 | AACCCCGp | AACCCCGp | 2224.3 |
| 650.4 | CGp | CGp | 650.4 |
| 17722.7 | AAAAUAACAUCC- | AAAAUAACAUCCCAUUUA- | 17722.7 |
| | CAUUUACUUCUUUA- | CUUCUUUAAACUAUUAA- | |
| | AACUAUUAAAACU- | AACUCAACCUCACAAAU- | |
| | CAACCUCACAAAU- | CACGp | |
| | CACGp | | |
| 15253.3 | CUAAACAAUACCAA- | CUAAACAAUACCAACUA- | 10103.1 |
| | CUAAUUCCACUU- | AUUCCACUUUUCCAGp | — |
| | UUCCAAAAAAUAAA- | AAAAUAAAAUUACACGp | 5166.2 |
| | AUUACACGp | | |
| 3566.2 | AAAAACUAACGp | AAAAACUGp | 2602.6 |
| | | ACGp | 979.6 |
| 7303.4 | ACCACUUCCAACCU- | ACCACUUCCAACCUAAUAC | 7303.4 |
| | AAUACAAAGp | AAAGp | |
| 1991.3 | AAAAAGp | AAAAAGp | 1991.3 |

### Example 3

### Analysis of Clinical Specimens

In order to show the applicability of the method according to the invention to the analysis of clinical problems, several colon specimens were additionally investigated. For this purpose, two tumor DNA specimens with a high degree of methylation and two normal colon specimens with a low degree of methylation were selected. For each of these, 10 clones of the amplified promoter region of the CDH13 gene were analyzed as described under Example 2 and compared with sequencing data (FIG. 5). A very good correlation is shown between the two methods both for the predominantly methylated (T1, T2) specimens as well as also for the predominantly unmethylated (N1, N2) specimens. The sequencing was in part not able to detect the methylation state in positions 32, 258 and 269. These positions are found either in the vicinity of the sequencing primer or at the end of the sequence. The limited measurement range of the MALDI spectrometer which was used, on the other hand, did not permit a clear assignment of all CpG positions. Thus, the absence of a fragment cannot absolutely be interpreted as an absence of methylation at the investigated position; this statement is justified by the detection of a longer fragment.

In clones A and J of specimen T1, the presence of the fragment 6+7 (m/z=5117) is caused by a methylation at positions 122 and 138, which frame the unmethylated position 136. In the case when several adjacent CpG positions are unmethylated, the resulting fragments will be larger and thus more difficult to detect. Thus, positions 154 and 210 appear to be non-analyzable, since the corresponding fragments are either so large that they can no longer be reliably detected, or so small that they can no longer stand out from the background noise. This does not represent, however, a basic limitation of the applicability of the method according to the invention. In the meantime MALDI devices have become known, which can analyze RNA up to a length of 2180 nucleotides and which can sequence RNA or DNA fragments in the length of 50 to 100 nucleotides (Berkenkamp et al., Infrared MALDI mass spectrometry of large nucleic acids. Science, 281, 260-262, 1998; Little et al. Verification of 50- to 100-mer DNA and RNA sequences with high-resolution mass spectrometry. Proc Nati Acad Sci USA, 92, 2318-2322, 1995).

### Example 4

### Direct Analysis of Clinical Specimens

Finally, an aliquot of the bisulfited colon DNA specimens was investigated directly (without prior cloning). For this purpose, first of all, a standard made from different mixtures of methylated and unmethylated DNA (0, 20, 40, 50, 60, 80, 100% methylated) was prepared and analyzed (FIG. 6). As expected, a reduced amount of methylation leads to a reduced intensity of the detected fragments. This, of course, does not apply to the control fragment (m/z=1991), which is formed independently of the degree of methylation and thus can be used for normalizing the signal. In comparison to the standard, the clinical specimens show different amounts of intensity. This can be attributed to the fact that several adjacent CpG positions have a greater comethylation than others. This has already resulted from the analysis of the clones (see above). Thus, the intense signal for fragments 6, 8, 9, 13 and 14 in the tumor specimen T1 shows a relatively high degree of comethylation in positions 122, 136, 138, 145, 152, 154, 258 and 269. These involve exactly the positions which have a comethylation in most of the analyzed cases (FIG. 5). The normalized relative intensities show a minimum of 50% methylation in these positions. In contrast, the absence of a signal or the presence of only a weak signal for fragments 1, 3, 4 and 5 is to be attributed to the fact that at positions 32, 81, 96 and 104, only a small degree of comethylation is present. These observations correspond to the clone data of Example 2. A similar methylation pattern was found for the tumor specimen T2. The lower intensity corresponds very well to the only small number of clones that show a comethylation in this specimen. The normal colon specimens N1 and N2 do not show a comethylation either in the direct analysis or in the clone analysis. Overall, the results of the direct analysis of the clinical specimens correlate very well with those of the clone analysis.

The comethylation of promoter regions is of decisive importance for many clinical problems. As shown in FIG. 6, the method according to the invention can detect the presence of comethylations in two or more adjacent positions. It selectivity represents a large advantage in comparison to direct bisulfite sequencing. It cannot, however, differentiate between specific methylation patterns and random methylation without clinical significance (see: Song et al.: Hypermethylation trigger of the glutathione-S-transferase gene (GSTP1) in prostate cancer cells. Oncogene, 21, 1048-1061, 2002).

### Example 5

### Combination of Allele-Specific Amplification and T1-RNase Characterization

Sequences of the Homo sapiens v-erb-b2 erythroblastic leukemia viral oncogene homolog 2 gene (Pub-Med Reference number: NM_{—}004448) will be investigated. For this purpose, DNA was produced by a “molecular displacement amplification”. Since only cytosine, but not methylcytosine, is incorporated in the amplification, this DNA is poor in 5-methylcytosine. A part of this DNA was subsequently treated by means of the Sssl methylase. A completely methylated DNA is thus formed. Subsequently, the DNA was bisulfited and amplified with a polymerase chain reaction in a sequence-specific manner. In this case, primers were used which contained nucleotides in their sequence that only occurred in a bisulfite strand of the originally methylated DNA. These primers thus amplified only bisulfited, methylated DNA. The following primers were utilized:

(Seq ID 8)

TCTTTTTCATATACGTGTGGGTATAAAATC;

(Seq ID 9)

GTAATACGACTCACTATAGGGAGCAAAaaTCAaaCAaCAACGA.

These primers were each mixed in a final concentration of 0.25 μmol/l with 1× Qiagen HotStar buffer, 0.2 mmol/l dNTPs (each dNTP), 0.04 U/μl of HotStarTaq from Qiagen in 25 μl, each containing 10 ng of DNA template, and processed with PCR. The following PCR program was used for this purpose: 95° C., 15 min; 95° C., 1 min; 55° C., 45 s; 72° C., 1:30 min.; 72° C., 10 min; 41 repetitions. These PCR products were analyzed on an agarose gel (see FIG. 7). After the PCR reaction, 10 μl of the PCR mix were mixed with 15 μl of transcription mix. This mix was constituted such that the following final concentrations were used in a 25 μl reaction: 1×MBI Fermentas T7 buffer, 0.8 U/μl of T7 RNA polymerase, 0.5 mmol/l NTPs (each one). This mixture was incubated for 1 h at 370 and then 1 μl of T1-RNAse [50 U/μl] was added. After the addition, it was incubated again for 1 h at 37°. Following this, the reaction batch was investigated in a mass spectrometer as described above. A spectrum produced in this way is shown in FIG. 8. Table 4 shows the masses expected in the case of complete methylation and the masses detected in the measurement. All theoretically predicted masses, which were larger than 1000 Da, were detected for the case of complete methylation. The investigated sequence was completely methylated. This was to be expected after the treatment with Sssl methylase. The mass of 1991.2 Da AAAAAGp, which resulted from the 5′ tail of the G-rich primer, showed the complete transcription of the PCR product.

TABLE 4

Fragments and their m/z values of the RNA from Example
3* after a digestion with RNase-T1.

| | | |
|---|---|---|
| Label | Mass | Sequence |

| | | |
|---|---|---|
| n.d. | 345.209 | Gp |
| n.d | 345.209 | Gp |
| n.d | 345.209 | Gp |
| n.d. | 674.418 | AGp |
| 7 | 6127.806 | CAAAAAUCAAACAACAACGp |
| 4 | 5071.058 | ACUUACUUCCAAAACGp |
| n.d | 979.602 | ACGp |
| 8 | 12362.446 | UCAAAACUUCUCUAAACACAUUACUAAAAUAACAUUUCGp |
| 5 | 5354.188 | UAUCUAAACCUUCUACGp |
| 2 | 3495.199 | CAUACACAUCGp |
| n.d. | 650.393 | CGp |
| 6 | 5425.277 | ACUACAUAAAAUUUACGp |
| 3 | 5048.019 | AUUUUAUACCCACACGp |
| n.d. | 1922.134 | UAUAUGp |
| 1 | 1991.254 | AAAAAGp |
| n.d. | 267.244 | A |

*sic; Example 5?- Trans. Note.

### BRIEF DESCRIPTION OF THE FIGURES

FIG. 1 shows schematically the principle of a particular embodiment of the method according to the invention. A promoter is introduced into the chemically converted DNA and a C-rich RNA is transcribed from this. A methylation-specific fragmentation pattern is produced by means of a T1-RNase digestion.

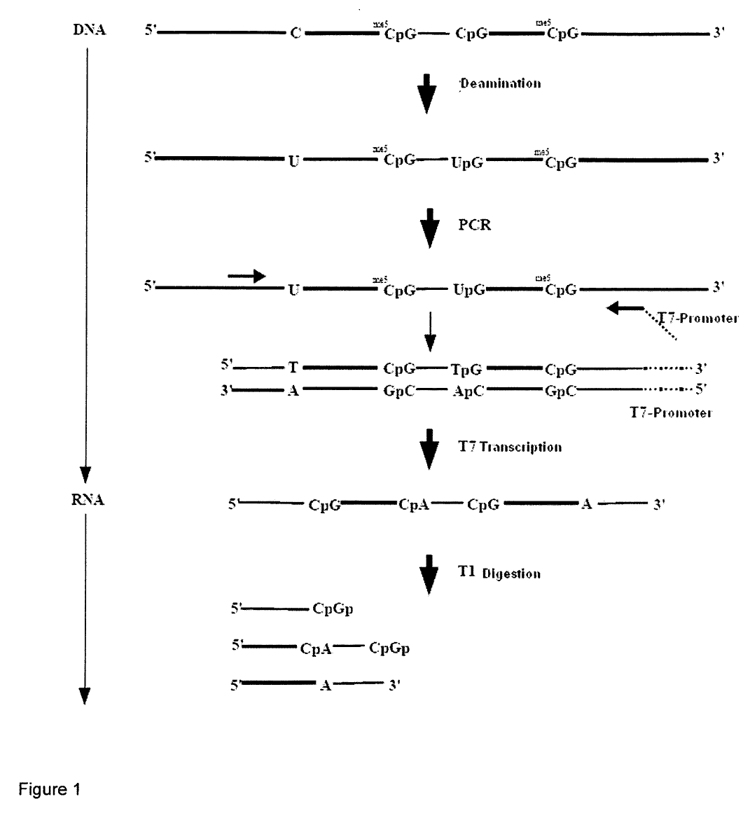

FIG. 2 shows schematically the principle of the embodiment according to the invention described in FIG. 1, with the additional use of a “control tag”.

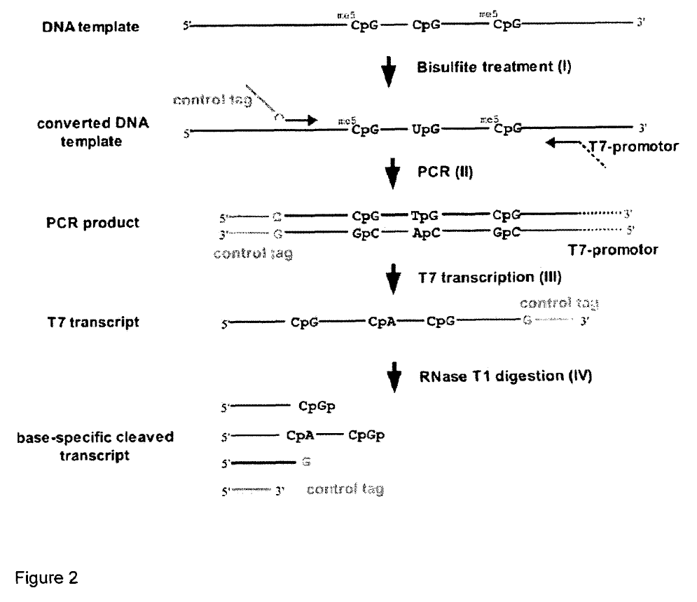

FIG. 3 shows the MALDI-TOF mass spectrum of the transcript of the synthetically methylated APC gene, which is digested with RNase-T1 (Example 1). The numbering of the peaks corresponds to that of Table 2.

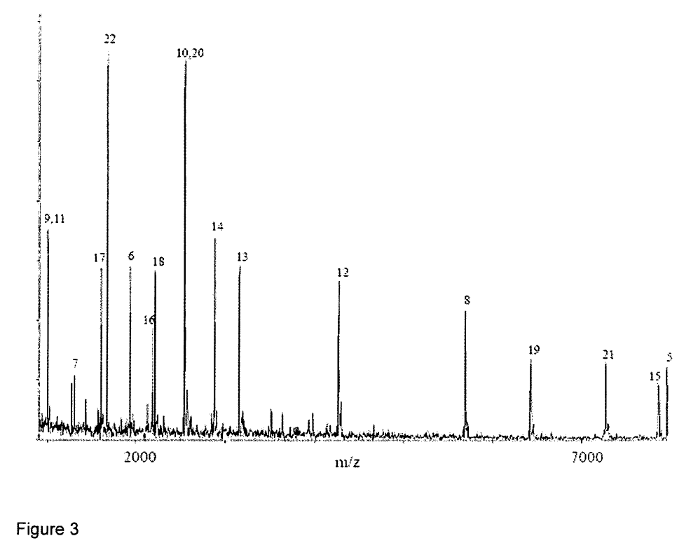

FIG. 4 shows the MALDI-TOF mass spectrum of Example 2.

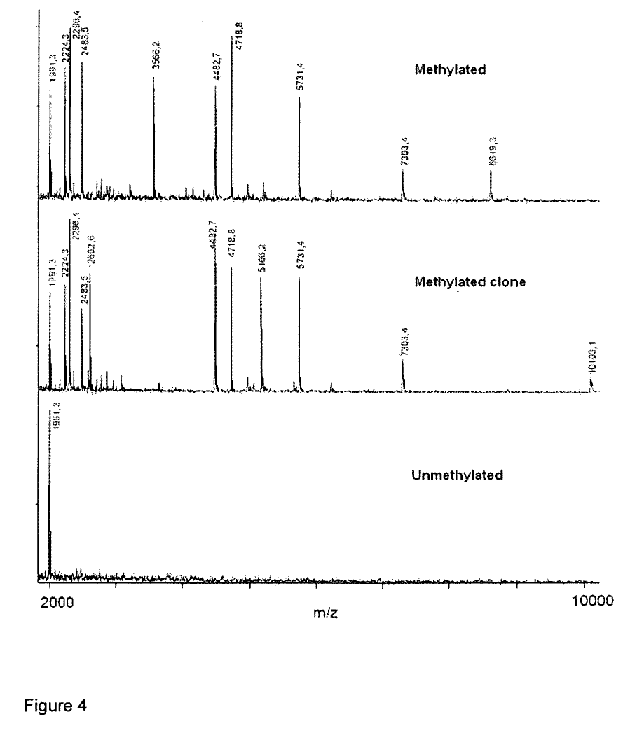

FIG. 5 shows the result of Example 3. CpG methylations in 10 clones (A-J) from two bisulfite-converted colon tumor DNA specimens (T1, T2) and two normal colon DNA specimens (N1, N2) were analyzed. Shown are the results after RNA cleavage and MALDI-TOF (left) or sequencing (right). The black circles characterize the methylated CpG positions, the white circles characterize the unmethylated CpG positions, the gray circles characterize fragments which were not clearly assignable, and the crosses characterize CpG positions which were not accessible to the analysis.

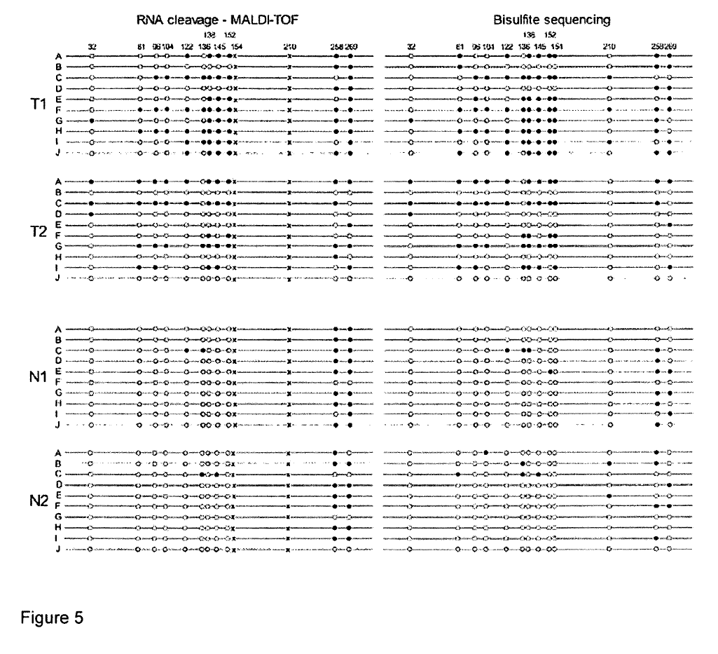

FIG. 6 shows the result of Example 4. A direct analysis was conducted of two bisulfite-converted colon tumor DNA specimens (T1, T2) and two normal colon DNA specimens (N1, N2) by means of a PCR, an in vitro transcription, an RNAse-T1 cleavage and a subsequent MALDI-TOF analysis. As a comparison, the DNA mixtures of a standard (top: mass spectrum of the specimens T1, T2, N1 and N2; bottom: spectrum of the DNA mixtures with different defined degrees of methylation.)

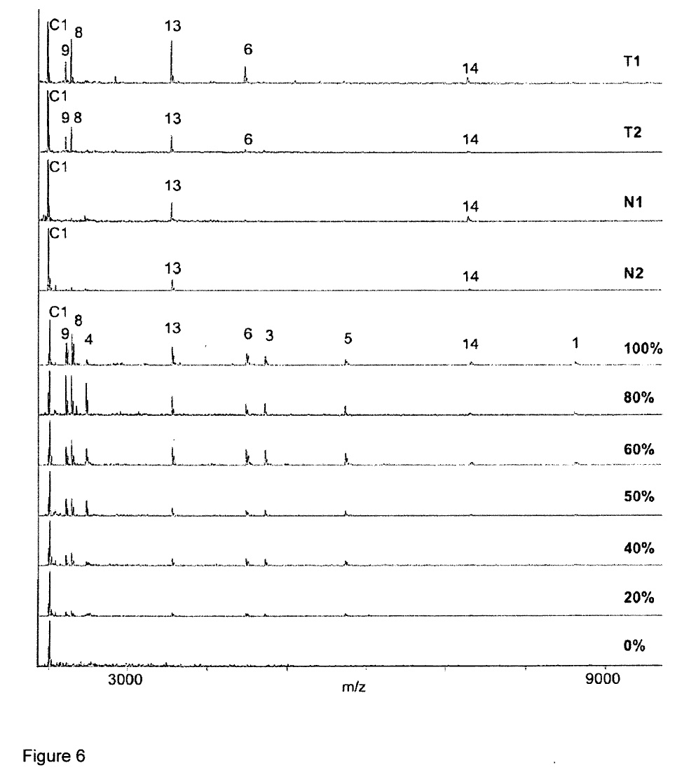

FIG. 7 shows the agarose gel of Example 3′. Shown is the amplification of bisulfited DNA of methylated and unmethylated DNA by means of methylation-specific T7 domain primers. The primers are selected such that they do not form a product from unmethylated DNA on genomic DNA and on bisulfited DNA. Bisulfited DNA treated with Sssl methylase, however, can be amplified.

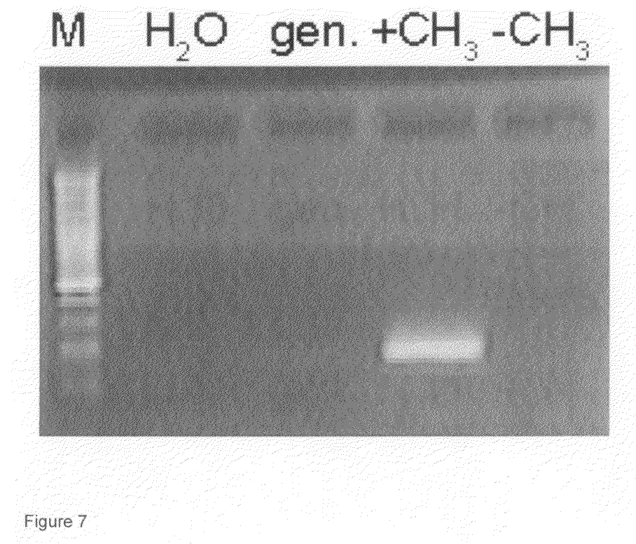

FIG. 8 shows the MALDI-TOF spectrum of Example 3*.

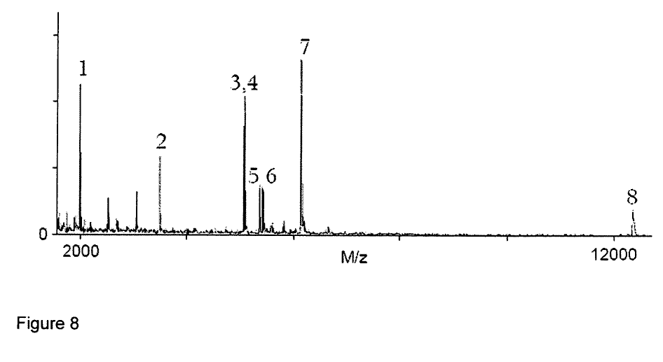

Table 1 is reproduced below:

| | | | | Inventive method: DNA-NASBA for sensitive | |
|---|---|---|---|---|---|
| | DNA-NASBA prior art | | | detection of DNA methylation | |
| # | Name of Step | Component | Function | Component | Function |
| 1 | Preparation | Specimen to be analyzed | The extraction of the DNA | As described in the | As described in the |
| | of template | Commercially obtainable DNA | makes sure that the latter | DNA-NASBA prior art | DNA-NASBA prior art |
| | DNA | extraction kit or respective | is present in adequate | | |
| | | individual components for an | purity and is accessible to | | |
| | | in-house protocol (“homebrew”) | the subsequent enzymatic | | |
| | | The DNA is extracted from the | reactions. | | |
| | | specimens to be examined | | | |
| | | corresponding to the | | | |
| | | manufacturer's instructions or the | | | |
| | | respective protocol. | | | |
| 2 | | | | Extracted DNA | Differences in |
| | | | | Reagents for the | methylation of |
| | | | | bisulfite conversion of the | cytosines (typically in |
| | | | | DNA (typically Na salts of | the sequence context |
| | | | | the sulfite and disulfite, | of CpG in the human |
| | | | | radical traps, organic | genome) are |
| | | | | solvents, water). | translated into |
| | | | | Device for the heat | differences in bases |
| | | | | incubation of the reaction | due to bisulfite |
| | | | | vessel | conversion: |
| | | | | Suitable quantities of the | methylated cytosines |
| | | | | above-described. | are not affected; |
| | | | | components are mixed in | cytosines without |
| | | | | a suitable reaction vessel | methyl groups are |
| | | | | and incubated for a short | deaminated, which |
| | | | | time at high temperatures | leads to the formation |
| | | | | (typically at 95° C.) | of uracil, which is |
| | | | | (typically for 5 min) and | replaced by thymine |
| | | | | then incubated for a | in the PCR. |
| | | | | specific time (typically 5-7 h) | CpG positions thus |
| | | | | at intermediate | remain unchanged |
| | | | | temperatures (typically at | CpG dinucleotides, if |
| | | | | 50-65° C.). Following this, | the cytosines are |
| | | | | NaOH or Tris with a high | methylated, but are |
| | | | | pH (typically 9.5) is added | [changed] to TpG |
| | | | | for desulfonation and the | positions at CpG |
| | | | | mixture is incubated for a | positions that were |
| | | | | short time (approximately | previously |
| | | | | 20 min) at high | unmethylated. |
| | | | | temperatures (typically | |
| | | | | 95° C.). Subsequently, the | |
| | | | | reaction mixture is | |
| | | | | desalted. | |
| 3 | Denaturation of | Extracted DNA from #1 | Heating up the mixture of | Bisulfite-converted DNA | As described in the |
| | template DNA | DNA oligonucleotides (T7-tailed | all necessary nucleic acid | from #2 | DNA-NASBA prior art |
| | and addition | primer), which in their 5′ region | components should | DNA oligonucleotides | |
| | of | consist of a base sequence that | assure that the DNA | (T7-tailed primer), which | |
| | oligonucleotides | corresponds to the promoter | double helix and all | in their 5′ region consist of | |
| | | sequence of the T7 | secondary structures are | a base sequence that | |
| | | DNA-dependent RNA polymerase | decomposed, which is an | corresponds to the | |
| | | (T7DdRp), and in their 3′ region | important prerequisite for | promoter sequence of the | |
| | | consist of a sequence that is | the specific binding of the | T7 DNA-dependent RNA | |
| | | reverse-complementary to the | primers to their | polymerase (T7DdRp), | |
| | | sense strand of the target | reverse-complementary | and in their 3′ region | |
| | | sequence within the template DNA | sequences in the template | consist of a sequence | |
| | | (typically 15 to 30 bp). | DNA in the addition step at | that is | |
| | | DNA oligonucleotides (2^{nd} | 41° C. | reverse-complementary | |
| | | primer), which contain a base | Depending on the DNA | to the (+) strand of the | |
| | | sequence that is | composition, denaturing | target sequence within | |
| | | reverse-complementary to a target | by heating is an optional | the template DNA | |
| | | sequence (typically 15 to 30 bp) of | step, which is not | (typically 15 to 30 bp). | |
| | | the antisense strand within the | absolutely necessary. | The latter sequence | |
| | | template DNA and lies 50 to 500 bp | | (target sequence in the | |
| | | downstream of the target | | template) should contain | |
| | | sequence of the T7-tailed | | no CpG or TpG positions. | |
| | | oligonucleotide. | | DNA oligonucleotides | |
| | | If detection is provided by means | | (2^{nd} primer), which | |
| | | of a specific probe: Probe | | contain a base sequence | |
| | | oligonucleotides (typically | | that is | |
| | | Molecular Beacon, or LightCycler | | reverse-complementary | |
| | | probes) which contain sequences | | to a sequence region | |
| | | (typically 15 to 30 bp) that are | | (typically 15 to 30 bp) of | |
| | | reverse-complementary to a | | the (−) strand of the target | |
| | | sense region of the target DNA | | sequence within the | |
| | | which is bounded by the primers | | template DNA and lies 50 | |
| | | (one of the above-described | | to 500 bp downstream of | |
| | | primers on each side). | | the target sequence of | |
| | | NASBA reaction buffer (typically | | the T7-tailed | |
| | | containing Tris, MgCl₂, KCl, | | oligonucleotide. Here | |
| | | dithiothreitol, DMSO, each dNTP, | | also this target sequence | |
| | | each NTP) | | should contain no CpG or | |
| | | Device for the heat incubation of | | TpG positions. | |
| | | the reaction vessels | | DNA nucleotides | |
| | | Suitable quantities of the | | (blockers) which contain | |
| | | above-described components are | | a sequence that is | |
| | | mixed in a suitable reaction vessel | | reverse-complementary | |
| | | and incubated for a short time | | to a region in the (−) | |
| | | (typically for 2 min) at high | | strand of the target | |
| | | temperatures (typically 95° C.) and | | sequence which has TpG | |
| | | then incubated for a short time | | positions and is typically | |
| | | (typically for 2 min) at intermediate | | 4-30 bp long. In addition, | |
| | | temperatures (typically 41° C). | | these blockers are | |
| | | | | protected by a | |
| | | | | modification of their 3′ | |
| | | | | end prior to extension. | |
| | | | | Most preferably, this | |
| | | | | protection involves a | |
| | | | | phosphorylation. This | |
| | | | | sequence can overlap | |
| | | | | with the sequence of the | |
| | | | | 2^{nd} primer. | |
| | | | | If detection is provided | |
| | | | | by means of a specific | |
| | | | | probe: Probe | |
| | | | | oligonucleotides (typically | |
| | | | | Molecular Beacon, or | |
| | | | | LightCycler probes which | |
| | | | | contain sequences | |
| | | | | (typically 15 to 30 bp) that | |
| | | | | are | |
| | | | | reverse-complementary | |
| | | | | to a (+) region of the | |
| | | | | target DNA which is | |
| | | | | bounded by the primers | |
| | | | | (one of the | |
| | | | | above-described primers | |
| | | | | on each side) and has 1-4 | |
| | | | | CpG dinucleotides. | |
| | | | | NASBA reaction buffer | |
| | | | | Device for the heat | |
| | | | | incubation of the reaction | |
| | | | | vessels | |
| | | | | Appropriate amounts of | |
| | | | | components described | |
| | | | | above are mixed in a | |
| | | | | suitable reaction vessel | |
| | | | | and incubated for a short | |
| | | | | time (typically 2 min) at | |
| | | | | high temperatures | |
| | | | | (typically 95° C.) and | |
| | | | | subsequently for a short | |
| | | | | time (typically 2 min) at | |
| | | | | medium temperatures | |
| | | | | (typically 41° C.). | |
| 4 | Extension of the | Reaction mixture from #3 | The T7-tailed primer is | As described in the | As described in the |
| | T7-tailed primer | Reverse transcriptase (RT) | extended by the RT, and | DNA-NASBA prior art | DNA-NASBA prior art |
| | by RT | (typically from “avian | in this way, a (−) DNA copy | | DNA templates are |
| | | myeloblastosis virus”) | of the sense strand is | | considered on the |
| | | Device for the incubation of the | prepared. | | basis of the |
| | | reaction vessel at appropriate | If a denaturing did not | | composition of the |
| | | temperature (typically 41° C.) | have to be conducted in # | | T7-tailed primer, |
| | | Appropriate quantities of RT are | 3, now the enzymes which | | which detects no CpG |
| | | added to the reaction mixture and | are described in # 6 can | | or TpG positions |
| | | the reaction vessel is incubated. | also be added. #5 is then | | independently from |
| | | | correspondingly omitted. | | their methylation |
| | | | | | state. |
| 5 | Denaturation of | Reaction mixture from #4 | Heating assures that the | As described in the | As described in the |
| | the reaction | Heating of the mixture to a high | newly prepared (−) DNA | DNA-NASBA prior art | DNA-NASBA prior art |
| | product | temperature (typically 95° C.), for a | copies of the template are | | |
| | | short time (typically 2 min). | denatured, which is a | | |
| | | | condition for the | | |
| | | | subsequent addition of the | | |
| | | | 2^{nd} primer in #6. | | |
| | | | This step can be omitted, | | |
| | | | depending on the specific | | |
| | | | target sequence; for the | | |
| | | | case when the sequence | | |
| | | | composition permits the | | |
| | | | addition of the 2^{nd} primer | | |
| | | | without prior denaturing, | | |
| | | | the enzymes which are | | |
| | | | described in # 6 can be | | |
| | | | added already in # 3. | | |
| 6 | Addition and | Denatured reaction mixture from | The second primer is | As described in the | As described in the |
| | extension of the | #5 | added to the copied (−) | DNA-NASBA prior art | DNA-NASBA prior art |
| | second primer | T7 DdRp, RNase-H | DNA strand and is | | If the target sequence |
| | | Appropriate quantities of the | extended by the RT, | | of the blocker |
| | | above-named enzymes are added | whereby a double strand | | overlaps with the 2^{nd} |
| | | to the reaction mixture and | is generated. | | primer, the extension |
| | | incubated for a relatively long time | | | of the (−) DNA copies, |
| | | (typically 90 min) at intermediate | | | which represent the |
| | | temperatures (typically 41° C.). | | | unmethylated state, is |
| | | | | | hindered. |
| | | | | | Otherwise, DNA |
| | | | | | templates are |
| | | | | | considered on the |
| | | | | | basis of the |
| | | | | | composition of the 2^{nd} |
| | | | | | primer, which detects |
| | | | | | no CpG or TpG |
| | | | | | positions |
| | | | | | independently from |
| | | | | | their methylation |
| | | | | | state. |
| 7 | Transcription of | Done in the reaction mixture and | T7-RNA polymerase binds | As described in the | As described in the |
| | the amplificate | during the incubation in step # 6. | to the double-stranded | DNA-NASBA prior art | DNA-NASBA prior art |
| | by the T7-RNA | | T7-promoter sequence | | |
| | polymerase | | and generates multiple | | |
| | | | RNA copies of the (−) | | |
| | | | strand. | | |
| 8 | | | | Done in the reaction | The blockers are |
| | | | | mixture and during the | added to the newly |
| | | | | incubation in step # 6. | prepared (−)-RNA |
| | | | | | copies which have |
| | | | | | TpG positions in their |
| | | | | | complementary |
| | | | | | region, but not to |
| | | | | | those which have |
| | | | | | CpG positions. In this |
| | | | | | way, an RNA-DNA |
| | | | | | heteroduplex is |
| | | | | | generated on RNA |
| | | | | | copies which |
| | | | | | represent the |
| | | | | | unmethylated state. |
| | | | | | The RNA within these |
| | | | | | heteroduplexes is |
| | | | | | digested by RNase-H. |
| | | | | | RNA copies which |
| | | | | | represent the |
| | | | | | methylated state are |
| | | | | | not affected. |
| 9 | Addition of the | Done in the reaction mixture and | The second primer is | Analogous to what is | Analogous to what is |
| | 2^{nd} primer | during the incubation in step # 6. | added to the (−)-RNA | described in the | described in the |
| | to the (−)-RNA | | copies from #7 and is | DNA-NASBA prior art | DNA-NASBA prior art |
| | copy and | | extended by the RT. In this | | See step #6 |
| | extension | | way, an RNA-DNA | | |
| | thereof | | heteroduplex is | | |
| | | | generated. | | |
| 10 | Degradation of | Done in the reaction mixture and | The (−)-RNA strand in the | Analogous to what is | Analogous to what is |
| | the RNA strand | during the incubation in step # 6. | RNA-DNA heteroduplexes | described in the | described in the |
| | due to RNase-H | | is digested by RNase-H. | DNA-NASBA prior art | DNA-NASBA prior art |
| | activity | | | | |
| 11 | Addition of the | Done in the reaction mixture and | The T7-tailed primer is | Analogous to what is | Analogous to what is |
| | T7-tailed primer | during the incubation in step # 6. | added to the (+)-DNA copy | described in the | described in the |
| | to the (+)-DNA | | from #10 and is extended | DNA-NASBA prior art | DNA-NASBA prior art |
| | copy and | | by the RT, whereby a | | |
| | extension | | double-stranded DNA is | | |
| | thereof | | generated. | | |
| 12 | Entry into the | Done in the reaction mixture and | See step #7. | Analogous to what is | Analogous to what is |
| | cyclic phase: | during the incubation in step # 6. | | described in the | described in the |
| | step #6 | | | DNA-NASBA prior art | DNA-NASBA prior art |
| | | | | | The amplification |
| | | | | | process for the most |
| | | | | | part results in the |
| | | | | | generation of RNA |
| | | | | | copies which |
| | | | | | represent the |
| | | | | | methylated state. |
| 13 | Detection of the | Done in the reaction mixture and | The probe is added to the | Analogous to what is | The probe is added to |
| | (−)-RNA copies | during the incubation in step # 6. | (−)-RNA copies from #7 | described in the | (−)-RNA copies, which |
| | by means of a | Device for the fluorescence | and #12. A fluorescent | DNA-NASBA prior art | represent the |
| | specific probe | detection of the reporter dye. | signal is generated. | | methylated state. A |
| | | | Continuous determination | | fluorescent signal is |
| | | | of the RNA copy number | | generated. |
| | | | by measurement of the | | Continuous |
| | | | generated fluorescence | | determination of the |
| | | | intensities. | | RNA copy number by |
| | | | | | measurement of the |
| | | | | | generated |
| | | | | | fluorescence |
| | | | | | intensities. |

1. A method for the analysis of cytosine methylations, hereby characterized in that
a) the DNA to be investigated is reacted so that 5-methylcytosine remains unchanged, while unmethylated cytosine is converted to uracil or to another base which differs from cytosine in its base-pairing behavior,
b) a promoter sequence is introduced into the DNA,
c) RNA is transcribed,
d) the RNA is analyzed,
e) a conclusion with regard to the methylation state of the DNA is made.

2. The method according to claim 1, further characterized in that in step b), the promoter sequence is ligated to the DNA.

3. The method according to claim 1, further characterized in that in step b), a PCR is carried out, in which one of the primers bears a promoter sequence.

4. The method according to claim 1, further characterized in that in step b), an NASBA or another amplification method based on transcription is utilized.

5. The method according to claim 1, further characterized in that T3, T7 or SP6 promoters are used as promoters.

6. The method according to claim 1, further characterized in that the analysis of the RNA in step d) is conducted by means of a hybridization on an oligomer array.

7. The method according to claim 1, further characterized in that the analysis of the RNA in step d) is performed in a mass spectrometer.

8. A method for the analysis of cytosine methylations in DNA, characterized in that the following steps are conducted:
a) the DNA to be investigated is reacted so that 5-methylcytosine remains unchanged, while unmethylated cytosine is converted to uracil or to another base which differs from cytosine in its base-pairing behavior,
b) the converted DNA is amplified by means of an amplification method based on transcription,
c) the amplificates are analyzed,
d) the methylation state of the investigated DNA is concluded.

9. A method for the analysis of cytosine methylations in DNA, characterized in that the following steps are conducted:
a) the DNA to be investigated is reacted so that 5-methylcytosine remains unchanged, while unmethylated cytosine is converted to uracil or to another base which differs from cytosine in its base-pairing behavior,
b) the converted DNA is amplified by means of an amplification method based on transcription, wherein the amplification occurs in the presence of at least one methylation-specific blocker molecule, which binds specifically to the background nucleic acid and hinders the amplification thereof,
c) the amplificates are analyzed,
d) the methylation state of the investigated DNA is concluded.

10. The method according to claim 9, further characterized in that the blocker molecules form DNA-RNA hybrids with the background RNA, the RNA part of which is decomposed in the course of the amplification cycle.

11. The method according to claim 9, further characterized in that the blocker involves an oligonucleotide which bears at least one methylation-specific dinucleotide.

12. The method according to claim 9, further characterized in that the amplificates are detected by means of real-time probes.

13. The method according to claim 1, further characterized in that the RNA is chemically or enzymatically fragmented prior to the analysis in step d).

14. The method according to claim 13, further characterized in that the fragmenting is conducted as a function of the methylation pattern of the investigated DNA.

15. The method according to claim 14, further characterized in that the fragmenting is conducted by means of the enzyme RNase-T1.

16. The method according to claim 14, further characterized in that the analysis of the fragments is conducted by means of MALDI-TOF, by means of electrophoretic methods or by means of chromatographic methods.

17. The method according to claim 14, further characterized in that, in addition to the promoter, control sequences are additionally introduced into the DNA, and these form the basis for being able to examine whether the fragmenting is complete.

18. Use of the method according to claim 1 for the diagnosis or prognosis of cancer disorders or other diseases associated with a modification of the cytosine methylation state, for predicting undesired drug effects, for establishing a specific drug therapy, for monitoring the result of a drug therapy, for distinguishing cell types or tissues and for investigating cell differentiation.

19. A kit, which consists of a bisulfite reagent and of at least one primer which bears a promoter sequence.

20. A kit according to claim 19, which additionally contains enzymes and/or other components for conducting an amplification method based on transcription.

21. A kit according to claim 20, which additionally contains at least one methylation-specific blocker oligomer.

22. A kit, which consists of a bisulfite reagent, primers and an enzyme which cleaves RNA in a nucleotide-specific manner, and, optionally, a polymerase and other reagents necessary for an amplification.

23. The method according to claim 8, further characterized in that the amplificates are detected by means of real-time probes.
